(19) United States
(12) Patent Application Publication
(10) Pub. No.: US 2018/0161449 A1
(43) Pub. Date: Jun. 14, 2018

(54) FORMATION OF CYCLOSPORIN A/CYCLODEXTRIN NANOPARTICLES

(71) Applicant: OCULIS EHF, Reykjavik (IS)

(72) Inventor: Thorsteinn LOFTSSON, Reykjavik (IS)

(21) Appl. No.: 15/577,883

(22) PCT Filed: May 27, 2016
(86) PCT No.: PCT/IB2016/000816

§ 371 Date: Nov. 29, 2017

Related U.S. Application Data
(60) Provisional application No. 62/168,492, filed on May 29, 2015.

(51) Int. Cl. A61K 47/69; A61K 9/00; A61K 9/16; A61P 27/04; A61K 38/13

(52) U.S. Cl. CPC A61K 47/6951; A61K 9/0048; A61K 9/1682; A61K 38/13; A61P 27/04; A61K 9/1652; A61K 9/1641

(57) ABSTRACT

Methods of forming cyclosporin/cyclodextrin complex nanoparticles and microparticles, and administration of the nano- and microsuspension formed to an eye of a human or animal in the form of aqueous eye drops suitable to elicit or enhance tear formation and for treatment of diseases of the eye and surrounding areas. The aqueous eye drop composition contains cyclosporin and a mixture of α-cyclodextrin and γ-cyclodextrin as well as one or more stabilizing polymers. α-Cyclodextrin solubilizes cyclosporin while γ-cyclodextrin promotes formation of cyclosporin/cyclodextrin complex aggregates. The polymers stabilize the aqueous nano- and microsuspension.

### BACKGROUND

The present invention relates to a novel aqueous eye drop composition wherein the active ingredient is cyclosporin A.

Topical administration of eye drops is the preferred means of drug administration to the eye due to the convenience and safety of eye drops in comparison to other routes of ophthalmic drug administration such as intravitreal injections and implants (Le Bourlais, C., Acar, L., Zia, H., Sado, P. A., Needham, T., Leverge, R., 1998. Ophthalmic drug delivery systems—Recent advances. Progress in Retinal and Eye Research 17, 33-58). Drugs are mainly transported by passive diffusion from the eye surface into the eye and surrounding tissues where, according to Fick's law, the drug is driven into the eye by the gradient of dissolved drug molecules. The passive drug diffusion into the eye is hampered by three major obstacles (Gan, L., Wang, J., Jiang, M., Bartlett, H., Ouyang, D., Eperjesi, F., Liu, J., Gan, Y., 2013. Recent advances in topical ophthalmic drug delivery with lipid-based nanocarriers. Drug Discov. Today 18, 290-297; Loftsson, T., Sigurdsson, H. H., Konradsdottir, F., Gisladottir, S., Jansook, P., Stefansson, E., 2008. Topical drug delivery to the posterior segment of the eye: anatomical and physiological considerations. Pharmazie 63, 171-179; Urtti, A., 2006. Challenges and obstacles of ocular pharmacokinetics and drug delivery. Adv. Drug Del. Rev. 58, 1131-1135).

First is aqueous drug solubility. Only dissolved drug molecules are able to diffuse into the eye and, thus, drugs must possess sufficient solubility in the aqueous tear fluid to diffuse into the eye. Increasing solubility of poorly soluble drugs through, for example, cyclodextrin complexation will increase their concentration gradient and their consequent passive diffusion into the eye (Loftsson, T., Järvinen, T., 1999. Cyclodextrins in ophthalmic drug delivery. Advanced Drug Delivery Reviews 36, 59-79).

The second obstacle is the rapid turnover rate of the tear fluid and the consequent decrease in concentration of dissolved drug molecules. Following instillation of an eye-drop (25-50 μl) onto the pre-corneal area of the eye, the greater part of the drug solution is rapidly drained from the eye surface and the tear volume returns to the normal resident volume of about 7 μl. Thereafter, the tear volume remains constant, but drug concentration decreases due to dilution by tear turnover and corneal and non-corneal absorption. The value of the first-order rate constant for the drainage of eye drops from the surface area is typically about 1.5 min⁻¹ in humans. Normal tear turnover is about 1.2 μl/min in humans and the pre-corneal half-life of topically applied drugs is between 1 and 3 minutes (Sugrue, M. F., 1989. The pharmacology of antiglaucoma drugs. Pharmacology & Therapeutics 43, 91-138). The precorneal half-life of topically applied drugs needs to be increased by, for example, formation of small drug/cyclodextrin microparticles in order to enhance their bioavailability (Johannesson, G., Moya-Ortega, M. D., Asgrimsdottir, G. M., Lund, S. H., Thorsteinsdottir, M., Loftsson, T., Stefansson, E., 2014. Kinetics of γ-cyclodextrin nanoparticle suspension eye drops in tear fluid. Acta Ophthalmologica 92, 550-556; Loftsson, T., Jansook, P., Stefansson, E., 2012. Topical drug delivery to the eye: dorzolamide. Acta Ophthalmologica 90, 603-608).

The third obstacle is slow drug permeation through the membrane barrier, i.e. cornea and/or conjunctiva/sclera. The drug molecules have to partition from the aqueous exterior into the membrane before they can passively permeate the membrane barrier. The result is that generally only few percentages of applied drug dose are delivered into the ocular tissues. The major part (50-100%) of the administered dose will be absorbed from the nasal cavity into the systemic drug circulation which can cause various side effects.

Dry eye syndrome is a common ocular disorder caused by decreased tear production that results in discomfort and visual disturbance. Dry eye syndrome has multifactorial etiology involving tear film instability, increased osmolality of the tear film and inflammation of the ocular surface, with potential damage to the ocular surface. Few therapies are available for this disease.

Cyclosporins are a group of peptides isolated from fungi of which cyclosporin A is best known. Numerous other natural and semi-synthesized cyclosporins exist including cyclosporin B, C, D, E, F, G and H (Lawen, A., 2015. Biosynthesis of cyclosporins and other natural peptidyl prolyl cis/trans isomerase inhibitors. Biochimica et Biophysica Acta 1850, 2111-2120; Peel, M., Sctiber, A., 2015. Semi-synthesis of cyclosporins. Biochimica et Biophysica Acta 1850, 2121-2144).

Cyclosporin A is a cyclic polypeptide drug obtained from the fermentation broth of two fungi, Trichoderma polysporum and Cylindrocarpon lucidum (Laupacis, A., Keown, P. A., Ulan, R. A., McKenzie, N., Stiller, C. R., 1982. Cyclosporin A: a powerful immunosuppressant. Canadian Medical Association Journal 126, 1041-1046). It has the molecular weight of 1202.6 Da, aqueous solubility of 0.008 mg/ml at ambient temperature and Log P_{octanol/water}=2.92 at 21° C. (El Tayar, N., Mark, A. E., Vallat, P., Brunne, R. M., Testa, B., Gunsteren, W. F. v., 1993. Solvent-dependent conformation and hydrogen-bonding capacity of cyclosporin A: evidence from partition coefficients and molecular dynamics simulations. J. Med. Chem. 36, 3753-3764; Loftsson, T., Hreinsdottir, D., 2006. Determination of aqueous solubility by heating and equilibration: A technical note. Aaps Pharmscitech 7, article number 4).

Cyclosporin A has a variety of biological activities, including immunosuppressive, anti-inflammatory and antifungal properties, the other cyclosporins having similar properties. In ophthalmology cyclosporin A has mainly been proven useful for patients with various inflammatory ocular surface disorders, including dry eye but it has also been used systemically to treat intraocular inflammatory and autoimmune diseases, such as uveitis. In 2003, 0.05% (w/v) cyclosporin A oil based eye drops (Restasis®; Alcon, Tex.) became commercially available for topical treatment of dry eye syndrome (Utine, C. A., Stern, M., Akpek, E. K., 2010. Clinical Review: Topical Ophthalmic Use of Cyclosporin A. Ocular Immunology & Inflammation 18, 352-361). However, using oils and surfactants to deliver cyclosporin A topically provides a low drug bioavailability and can cause blurry vision, burning sensation, itching and irritation of the conjunctiva. These side effects can be avoided by delivering cyclosporin A in the form of aqueous eye drops.

The aqueous solubility of cyclosporin A can be increased through formation of cyclodextrin complexation and the contact time of cyclosporin A with the eye surface can be increased through formation of micro- and nanoparticles. α-Cyclodextrin, methylated α-cyclodextrin and methylated β-cyclodextrin have been reported to improve aqueous solubility of cyclosporin A (Miyake, K., Arima, H., Irie, T., Hirayama, F., Uekama, K., 1999. Enhanced absorption of cyclosporin A by complexation with dimethyl-beta-cyclodextrin in bile duct-cannulated and -noncannulated rats. Biological & Pharmaceutical Bulletin 22, 66-72).

Cyclodextrins are cyclic oligosaccharides containing 6 (α-cyclodextrin), 7 (β-cyclodextrin) and 8 (γ-cyclodextrin) glucopyranose monomers linked via α-1,4-glycoside bonds. α-Cyclodextrin, β-cyclodextrin and γ-cyclodextrin are natural products formed by microbial degradation of starch. The outer surface of the doughnut shaped cyclodextrin molecules is hydrophilic, bearing numerous hydroxyl groups, but their central cavity is somewhat lipophilic (Kurkov, S. V., Loftsson, T., 2013. Cyclodextrins. Int J Pharm 453, 167-180; Loftsson, T., Brewster, M. E., 1996. Pharmaceutical applications of cyclodextrins. 1. Drug solubilization and stabilization. Journal of Pharmaceutical Sciences 85, 1017-1025). In addition to the three natural cyclodextrins numerous water-soluble cyclodextrin derivatives have been synthesized and tested as drug carriers, including cyclodextrin polymers (Stella, V. J., He, Q., 2008. Cyclodextrins. Tox. Pathol. 36, 30-42).

In aqueous solutions, cyclodextrins are able to form inclusion complexes with many drugs by taking up a drug molecule, or more frequently some lipophilic moiety of the molecule, into the central cavity. This property has been utilized for drug formulation and drug delivery purposes. Formation of drug/cyclodextrin inclusion complexes, their effect on the physicochemical properties of drugs, the ability of drugs to permeate biomembranes and usage of cyclodextrins in pharmaceutical products have been reviewed (Loftsson, T., Brewster, M. E., 2010. Pharmaceutical applications of cyclodextrins: basic science and product development. Journal of Pharmacy and Pharmacology 62, 1607-1621; Loftsson, T., Brewster, M. E., 2011. Pharmaceutical applications of cyclodextrins: effects on drug permeation through biological membranes” J. Pharm. Pharmacol. 63, 1119-1135; Loftsson, T., Järvinen, T., 1999. Cyclodextrins in ophthalmic drug delivery. Advanced Drug Delivery Reviews 36, 59-79).

Cyclodextrins are known to increase both chemical and physical stability of proteins and peptides in aqueous solutions. Furthermore, cyclodextrins are known to increase aqueous solubility of poorly soluble protein and peptide drugs (J. Horský and J. Pitha, Inclusion complexes of proteins: interaction of cyclodextrins with peptides containing aromatic amino acids studies by competitive spectrophotometry. J. Inclusion Phenom. Mol. Recognit. Chem., 18, 291-300, 1994). Cyclodextrins and cyclodextrin complexes self-associate to form aggregates and the drug/cyclodextrin complex aggregates have been formulated as drug carriers (Bonini, M., Rossi, S., Karlsson, G., Almgren, M., Lo Nostro, P., Baglioni, P., 2006. Self-assembly of beta-cyclodextrin in water. Part 1: Cryo-TEM and dynamic and static light scattering. Langmuir 22, 1478-1484; He, Y., Fu, P., Shen, X., Gao, H., 2008. Cyclodextrin-based aggregates and characterization by microscopy. Micron 39, 495-516; Loftsson, T., 2014. Self-assembled cyclodextrin nanoparticles and drug delivery. J Incl Phenom Macro 80, 1-7; Messner, M., Kurkov, S. V., Jansook, P., Loftsson, T., 2010. Self-assembled cyclodextrin aggregates and nanoparticles. Int J Pharm 387, 199-208). Previously it has been shown that the drug/cyclodextrin aggregates enhance topical drug delivery to the eye (Thorsteinn Loftsson and Einar Stefansson, Cyclodextrin nanotechnology for ophthalmic drug delivery, U.S. Pat. No. 7,893,040 (Feb. 22, 2011); Thorsteinn Loftsson and Einar Stefansson, Cyclodextrin nanotechnology for ophthalmic drug delivery, U.S. Pat. No. 8,633,172 (Jan. 21, 2014); Thorsteinn Loftsson and Einar Stefansson, Cyclodextrin nanotechnology for ophthalmic drug delivery U.S. Pat. No. 8,999,953 (Apr. 7, 2015)). Not all drugs, especially not all peptides and proteins, are able to form drug/cyclodextrin aggregates of sufficient size to be retained on the eye surface after topical administration.

It has now been surprisingly discovered that, while α-cyclodextrin is an excellent solubilizer of cyclosporins, addition of γ-cyclodextrin to aqueous cyclosporin/α-cyclodextrin solutions promotes formation of nano- and microparticles containing cyclosporin/cyclodextrin complexes.

### SUMMARY

In a first aspect, there is provided herein an aqueous ophthalmic composition comprising:
(a) a cyclosporin which is effective ophthalmologically;
(b) α-cyclodextrin in an amount effective to form a water-soluble complex with said cyclosporin;
(c) γ-cyclodextrin in an amount effective to produce formation of cyclosporin/α-cyclodextrin complex aggregates;
(d) cyclosporin/cyclodextrin particles with diameters of from about 100 nm to about 100 μm, comprising both said α-cyclodextrin, and said γ-cyclodextrin;
(e) water; and
(f) optionally, a polymeric stabilizing agent;
   the total concentration of said cyclosporin in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from 1% (w/v) to about 25% (w/v), and the total fraction of cyclosporin in particles with diameters greater than about 300 nm being not less than about 10%.

In a second aspect, there is provided herein a method of eliciting or inducing or enhancing tear formation in a subject in need thereof, said method comprising topically administering to the eye or eyes of said subject an amount of a composition as defined in the preceding paragraph effective to elicit or induce tear formation.

In yet a third aspect, there is provided herein an aqueous ophthalmic composition comprising:
(a) an amount of cyclosporin A which is effective ophthalmologically;
(b) α-cyclodextrin, in an amount effective to form a water-soluble complex with cyclosporin A;
(c) γ-cyclodextrin in an amount effective to produce formation of cyclosporin A/α-cyclodextrin complex aggregates;
(d) cyclosporin/cyclodextrin particles with diameters of from about 100 nm to about 100 μm, comprising both said γ-cyclodextrin and said γ-cyclodextrin;
(e) water; and
(f) optionally, a polymeric stabilizing agent
   the total concentration of said cyclosporin A in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from about 1 (w/v) to about 25% (w/v) and the total fraction of cyclosporin A in particles with diameters greater than about 300 nm being not less than about 10%.

In still a fourth aspect, there is provided herein a method of forming agglomerates of a cyclosporin, especially cyclosporin A, said method comprising solubilizing a therapeutically effective cyclosporin, especially cyclosporin A, in water in a quantity of α-cyclodextrin sufficient to essentially completely dissolve said cyclosporin, and adding sufficient γ-cyclodextrin to form cyclosporin (especially cyclosporin A)/α-cyclodextrin complex aggregates, optionally with a polymeric stabilizing agent, to produce cyclosporin (especially cyclosporin A)/cyclodextrin particles with diameters of from about 100 nm to about 100 μm, comprising both said α-cyclodextrin and said γ-cyclodextrin. The term “essentially completely” herein and throughout this application means at least 75% to about 100% dissolved. In exemplary embodiments, this can mean at least about 75%, at least about 80%, at least about 85%, at least about 90%, at least about 95%, and in a preferred embodiment is at least about 90%.

In still a fifth aspect, there is provided an aqueous ophthalmic composition comprising:
(a) an amount of a cyclosporin, preferably cyclosporin A, which is therapeutically effective ophthalmologically;
(b) α-cyclodextrin in an amount effective to form a water-soluble complex with said cyclosporin;
(c) γ-cyclodextrin in an amount effective to produce formation of cyclosporin/α-cyclodextrin complex aggregates;
(d) cyclosporin/cyclodextrin particles with diameters of from about 100 nm to about 100 μm, comprising both said α-cyclodextrin and said γ-cyclodextrin;
(e) water; and
(f) optionally, a polymeric stabilizing agent;
   the total concentration of said cyclosporin in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from about 1% (w/v) to about 25% (w/v), the total fraction of cyclosporin in particles with diameters greater than about 300 nm being not less than about 10%, for use in the eliciting or enhancing of tear formation by topical administration of an effective amount thereof to the eye or eyes of a subject in need of same.

Still further, in a sixth aspect, there is provided herein use of cyclosporin in the manufacture of an aqueous ophthalmic composition comprising:
(a) an amount of a cyclosporin, preferably cyclosporin A, which is therapeutically effective ophthalmologically;
(b) α-cyclodextrin in an amount effective to form a water-soluble complex with said cyclosporin;
(c) γ-cyclodextrin in an amount effective to produce formation of cyclosporin/α-cyclodextrin complex aggregates;
(d) cyclosporin/cyclodextrin particles with diameters of from about 100 nm to about 100 μm, comprising both said α-cyclodextrin and said γ-cyclodextrin;
(e) water; and
(f) optionally, a polymeric stabilizing agent;
   the total concentration of said cyclosporin in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from about 1% (w/v) to about 25% (w/v), the total fraction of cyclosporin in particles with diameters greater than about 300 nm being not less than about 10%, for eliciting tear formation by topical administration of an effective amount thereof to the eye or eyes of a subject in need of same.

### DETAILED DESCRIPTION

The patents, published applications and scientific literature referred to herein establish the knowledge of those with skill in the art and are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a method, the term “comprising” means that the method includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a method or composition; for example, steps or features or components which would significantly interfere with the desired properties of the compounds or compositions described herein, i.e., the method or composition is limited to the specified steps or materials and those which do not materially affect the basic and novel characteristics of the method or composition.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001).

As used herein, “treating” means reducing, hindering or inhibiting the development of, or controlling, inhibiting, alleviating and/or reversing one or more symptoms in the individual to which a composition as described herein has been administered, as compared to the symptoms of an individual not being administered the composition. A practitioner will appreciate that the compositions and methods described herein are to be used in concomitance with continuous clinical evaluations by a skilled practitioner (physician or veterinarian) to determine subsequent therapy. Such evaluation will aid and inform in evaluating whether to increase, reduce or continue a particular treatment dose, and/or to alter the mode of administration.

The methods described herein are intended for use with any subject/patient that may experience their benefits. Thus, the terms “subjects” as well as “patients,” “individuals” and “warm-blooded animals” and “mammals” include humans as well as non-human subjects, such as non-human animals that may experience the same or similar ocular disorders, in particular, dogs, horses and cats. In particular, these animals, like humans, can suffer from conditions in which too few tears are produced and can benefit from the instant method of eliciting or inducing tear formation.

The following definitions and explanations are also relevant to this application.

An ocular condition is a disease, ailment or other condition which affects or involves the eye, one of the parts or regions of the eye, or the surrounding tissues such as the lacrimal glands. Broadly speaking, the eye includes the eyeball and the tissues and fluids which constitute the eyeball, the periocular muscles (such as the oblique and rectus muscles), the portion of the optic nerve which is within or adjacent to the eyeball and surrounding tissues such as the lacrimal glands and the eye lids.

An anterior ocular condition is a disease, ailment or condition which affects or which involves an anterior (i.e. front of the eye) ocular region or site, such as a periocular muscle, an eye lid, lacrimal gland or an eye ball tissue or fluid which is located anterior to the posterior wall of the lens capsule or ciliary muscles.

Thus, an anterior ocular condition primarily affects or involves one or more of the following: the conjunctiva, the cornea, the anterior chamber, the iris, the posterior chamber (behind the retina but in front of the posterior wall of the lens capsule), the lens, or the lens capsule, and blood vessels and nerves which vascularize or innervate an anterior ocular region or site. An anterior ocular condition is also considered herein as extending to the lacrimal apparatus, in particular, the lacrimal glands which secrete tears, and their excretory ducts which convey tear fluid to the surface of the eye.

A posterior ocular condition is a disease, ailment or condition which primarily affects or involves a posterior ocular region or site such as the choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition such as, for example, macular degeneration (such as non-exudative age-related macular degeneration and exudative age-related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

An anterior ocular condition can include a disease, ailment or condition such as, for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

The present description is concerned with and directed to ophthalmic compositions for topical drug delivery to the eye(s) and to methods for the treatment of an ocular condition, such as an anterior ocular condition or a posterior ocular condition or an ocular condition which can be characterized as both an anterior ocular condition and a posterior ocular condition.

Dry eye syndrome (DES), also known as dry eye disease (DED), keratoconjunctivitis sicca (KCS), and keratitis sicca, is a common ocular condition caused by decreased tear production that results in discomfort and visual disturbance. Dry eye syndrome has multifactorial etiology involving tear film instability, increased osmolality of the tear film and inflammation of the ocular surface, with potential damage to the ocular surface. The therapy of dry eye depends on its severity. Artificial tears can provide temporary improvement in eye irritation and blurred vision symptoms. Corticosteroids can be used to decrease ocular surface inflammation. However, the most promising treatment against dry eye syndrome is topically administered cyclosporin A, which increases tear production and thus relieves inflammation. In clinical trials, the commercial 0.05% cyclosporin A/oily vehicle was effective in 15% of patients after 6 months, compared to 5% in placebo.

Cyclosporin A is a peptide that inhibits T-cell activation and consequently inhibits the inflammatory cytokine production (selective inhibition of IL-I). In addition, cyclosporin A inhibits apoptosis by blocking the opening of the mitochondrial permeability transition pore and by increasing the density of conjunctival goblet cells (Kunert, K. S., Tisdale, A. S., Gipson, I. K., 2002. Goblet cell numbers and epithelial proliferation in the conjunctiva of patients with dry eye syndrome treated with cyclosporine. Archives of Ophthalmology 120, 330-337). Conditions associated with dry eye can also benefit from topical administration of cyclosporin A. For example, refractive surgery of the cornea is almost contraindicated in patients with dry eye. Cyclosporin A treatment before and after surgery can help these patients obtain a surgical correction of their refractive error without the risk of dry eye. The marketed eye drops contain 0.05% (w/v) cyclosporin A ophthalmic emulsion (Restasis®; Alcon, Tex.). The eye drops are administered twice a day. However, using oils and surfactants to deliver cyclosporin A topically provides a low drug bioavailability and can cause blurry vision, a burning sensation, itching and irritation of the conjunctiva. These side effects can be avoided by delivering cyclosporin A in the form of aqueous eye drops. Furthermore, the therapeutic efficacy of cyclosporin A eye drops would increase if the cyclosporin A concentration in the eye drops can be increased by 10-fold, from 0.05% to 0.5% (w/v). Cyclosporin A is also known as ciclosporin or as cyclosporine.

### Microparticles for Ophthalmic Delivery

This description relates to enhanced topical peptide and protein drug delivery, particularly cyclosporin, especially cyclosporin A, delivery to the eye and the surrounding tissues obtained by maintaining the aqueous tear fluid saturated with the drug for an enhanced duration of time. When the tear fluid is saturated with the drug then the drug molecules have a maximum tendency to partition from the fluid into the cornea, conjunctiva/sclera and other tissues that are in contact with the tear fluid. These tissues are covered by lipophilic membranes. Passive drug diffusion through these membranes is driven by the gradient of chemical potential within the membrane and, thus, high drug concentration at the membrane surface will enhance drug delivery through the membranes and into the surrounding tissues. Under normal conditions drugs that are administered to the eye as aqueous eye drop solutions will rapidly be diluted and washed from the eye surface by the constant flow of tear fluid. Drug dilution on the eye surface reduces drug flow from the surface into the eye and surrounding tissues. Many ophthalmic drugs are poorly soluble compounds that do not display sufficient solubility in the aqueous tear fluid. Such drugs are sometimes administered as aqueous eye drop suspensions and this will result in somewhat sustained drug concentrations at the eye surface. However, due to their low water-solubility, their absorption from the eye surface will be dissolution rate limited, that is, drug absorption into the eye will be hampered by the slow dissolution of the solid drug. Administration of such lipophilic drugs as more water-soluble drug/cyclodextrin complexes does increase the dissolution rate of the solid drug in the tear fluid, preventing dissolution rate limited drug absorption. Particles in an ophthalmic eye drop suspension are washed more slowly from the eye surface than dissolved drug molecules, partly due to adhesion of the particles to the surrounding tissues. Enhanced absorption is obtained through introduction of more favorable physicochemical conditions for passive drug diffusion. Administration of the aqueous drug/cyclodextrin eye drop suspensions containing solid drug/cyclodextrin complexes will ensure constant high concentrations of dissolved drug in the aqueous tear fluid over an extended time period.

As noted in the BACKGROUND hereinabove, various pre-corneal factors will limit the ocular absorption by shortening corneal contact time of applied drugs. The most important factors are the drainage of the installed solution, non-corneal absorption and induced lacrimation. These factors, and the membrane barriers themselves, will limit penetration of a topically administered ophthalmic drug. As a result, only a few percentages of the applied dose are delivered into the intraocular tissues. The major part (50-100%) of the administered dose will be absorbed into the systemic blood circulation which can cause various side effects. Following instillation of an applied eye-drop (25-50 μl) onto the pre-corneal area of the eye, the greater part of the drug solution is rapidly drained from the eye surface and the solution volume returns to the normal resident tear volume of about 7 μl. Thereafter, the pre-ocular solution volume remains constant, but drug concentration decreases due to dilution by tear turnover and corneal and non-corneal absorption. The value of the first-order rate constant for the drainage of eye drops from the pre-corneal area is typically about 1.5 min⁻¹ in humans with a tear turnover rate of about 1.2 μl/min and, consequently, the precorneal half-life of topically applied drugs is only between 1 and 3 minutes after the initial eye drop drainage from the eye surface.

### Formation of Drug/Cyclodextrin Particles

Cyclodextrins and drug/cyclodextrin complexes are able to self-assemble in aqueous solutions to form nano-sized aggregates and micellar-like structures that are also able to solubilize poorly soluble drugs through non-inclusion complexation and micellar-like solubilization (Messner, M., Kurkov, S. V., Jansook, P., Loftsson, T., 2010. Self-assembled cyclodextrin aggregates and nanoparticles. Int J Pharm 387, 199-208). Cyclodextrins are known to solubilize cyclosporin A in aqueous solutions and aqueous cyclosporin A eye drop solutions have been described (Kanai, A., Alba, R. M., Takano, T., Kobayashi, C., Nakajima, A., Kurihara, K., Yokoyama, T., Fukami, M., 1989. The effect on the cornea of alpha cyclodextrin vehicle for cyclosporin eye drops. Transplant. Proc. 21, 3150-3152). Previously we have developed and tested cyclodextrin-based eye drops containing dexamethasone (Johannesson, G., Moya-Ortega, M. D., Asgrimsdottir, G. M., Lund, S. H., Thorsteinsdottir, M., Loftsson, T., Stefansson, E., 2014. Kinetics of γ-cyclodextrin nanoparticle suspension eye drops in tear fluid. Acta Ophthalmologica 92, 550-556; Tanito, M., Hara, K., Takai, Y., Matsuoka, Y., Nishimura, N., Jansook, P., Loftsson, T., Stefansson, E., Ohira, A., 2011. Topical dexamethasone-cyclodextrin microparticle eye drops for diabetic macular edema. Invest Ophth Vis Sci 52, 7944-7948) and dorzolamide (Johannesson, G., Moya-Ortega, M. D., Asgrimsdottir, G. M., Lund, S. H., Thorsteinsdottir, M., Loftsson, T., Stefansson, E., 2014. Kinetics of γ-cyclodextrin nanoparticle suspension eye drops in tear fluid. Acta Ophthalmologica 92, 550-556; Gudmundsdottir, B. S., Petursdottir, D., Asgrimsdottir, G. M., Gottfredsdottir, M. S., Hardarson, S. H., Johannesson, G., Kurkov, S. V., Jansook, P., Loftsson, T., Stefansson, E., 2014. γ-Cyclodextrin nanoparticle eye drops with dorzolamide: effect on intraocular pressure in man. J. Ocul. Pharmacol. Ther. 30, 35-41) and irbesartan (Muankaew, C., Jansook, P., Stefansson, E., Loftsson, T., 2014. Effect of γ-cyclodextrin on solubilization and complexation of irbesartan: influence of pH and excipients. Int J Pharm 474, 80-90) in cyclodextrin nanoparticles. The studies show that the nanoparticles increase the drug contact time with the ocular surface and the ocular bioavailability of the drugs.

This application relates to formulation of water based cyclosporin eye drops where α-cyclodextrin is used to increase the aqueous solubility of cyclosporin A and γ-cyclodextrin is used to form drug/cyclodextrin nano- and microparticles. Although α-cyclodextrin is able to solubilize cyclosporin A through formation of water-soluble cyclosporin A/α-cyclodextrin complexes, the complexes formed have little tendency to form nano- and microparticles. γ-Cyclodextrin has much less tendency to form complexes with cyclosporin A. However, it has been unexpectedly observed that addition of γ-cyclodextrin to an aqueous cyclosporin A/α-cyclodextrin complex solution promoted formation of cyclosporin A/cyclodextrin complex aggregates.

Although cyclosporin A eye drops are the main focus of this application, other lipid-soluble and poorly water-soluble peptide drugs can be included in the described cyclodextrin-based drug delivery system such as other cyclosporins, somatostatin and somatostatin analogs, and lipid-soluble peptide prodrugs.

The aqueous eye drop composition herein contains cyclosporin in a cyclodextrin complex containing a mixture of α-cyclodextrin and γ-cyclodextrin as well as one or more optional stabilizing polymers. The α-cyclodextrin solubilizes the cyclosporin while γ-cyclodextrin promotes formation of cyclosporin/cyclodextrin complex aggregates. At least one polymer stabilizes the aqueous nano- and microsuspension.

The polymeric stabilizing agent is selected from the group consisting of polyoxyethylene fatty acid esters, polyoxyethylene alkylphenyl ethers, polyoxyethylene alkyl ethers, cellulose derivatives (alkyl celluloses, hydroxyalkyl celluloses and hydroxyalkyl alkylcelluloses), carboxyvinyl polymers (i.e. carbomers such as Carbopol 971 and Carbopol 974), polyvinyl polymers, polyvinyl alcohols, and polyvinylpyrrolidones and related polymeric stabilizers indicated below.

Useful polymeric stabilizers include polyethyleneglycol monostearate, polyethyleneglycol monostearate, polyethyleneglycol distearate, hydroxypropyl methylcellulose, hydroxypropylcellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., Tween 20 and Tween 80 (ICI Specialty Chemicals)); polyethylene glycols (e.g., Carbowax 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics F68 and FI08, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508 (T-1508) (BASF Wyandotte Corporation), Tritons X-200, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, and the like.

Especially useful stabilizers are poloxamers. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate. Poloxamers are also referred to by their trade name Pluronic such as Pluronic 10R5, Pluronic 17R2, Pluronic 17R4, Pluronic 25R2, Pluronic 25R4, Pluronic 31R1, Pluronic F 108 Cast Solid Surfacta, Pluronic F 108 NF, Pluronic F 108 Pastille, Pluronic F 108NF Prill Poloxamer 338, Pluronic F 127, Pluronic F 127 NF, Pluronic F 127 NF 500 BHT Prill, Pluronic F 127 NF Prill Poloxamer 407, Pluronic F 38, Pluronic F 38 Pastille, Pluronic F 68, Pluronic F 68 Pastille, Pluronic F 68 LF Pastille, Pluronic F 68 NF, Pluronic F 68 NF Prill Poloxamer 188, Pluronic F 77, Pluronic F 77 Micropastille, Pluronic F 87, Pluronic F 87 NF, Pluronic F 87 NF Prill Poloxamer 237, Pluronic F 88, Pluronic F 88 Pastille, Pluronic F 98, Pluronic L 10, Pluronic L 101, Pluronic L 121, Pluronic L 31, Pluronic L 35, Pluronic L 43, Pluronic L 44 NF Poloxamer 124, Pluronic L 61, Pluronic L 62, Pluronic L 62 LF, Pluronic L 62D, Pluronic L 64, Pluronic L 81, Pluronic L 92, Pluronic L44 NF INH surfactant Poloxamer 124 View, Pluronic N 3, Pluronic P 103, Pluronic P 104, Pluronic P 105, Pluronic P 123 Surfactant, Pluronic P 65, Pluronic P 84 and Pluronic P 85.

The following EXAMPLES are detailed by way of illustration only and are not to be construed as limiting in spirit or in scope, many modification both in materials and in methods will be apparent to those skilled in the art.

### Example 1

The effect of cyclodextrins on the solubility of cyclosporin A in water was investigated. An excess amount of drug was added to aqueous solutions containing up to 20% (w/v) cyclodextrin. The solutions were sonicated at 40-50° C. for 45-60 minutes in sealed glass vials, and then allowed to cool to room temperature (22-23° C.). Small amount of solid drug was then added to each vial, the vial resealed and allowed to equilibrate under constant agitation and protected from light for 7 days at room temperature. When the solutions had reached equilibrium, they were filtered through a 0.45 μm membrane filter and analyzed by high pressure liquid chromatography. The apparent complexation constant for cyclosporin A/cyclodextrin complexes (K_{1:1}) was determined using the phase-solubility method developed by Higuchi and Connors (Higuchi, T., Connors, K. A., 1965. Phase solubility techniques. Advanced Analytical Chemistry of Instrumentation 4, 117-212.). The complexation efficiency (CE) was determined from the slope of phase-solubility diagrams (plots of total solubility of the drug versus total CD concentration in mol/l) where S₀ is the intrinsic solubility of the drug:

$$CE = \frac{Slope}{1 - Slope} = {K}_{1 : 1} \cdot {S}_{0}$$

TABLE 1

| | | | Solubility in | Solubility in |
|---|---|---|---|---|
| | | | the presence | the presence |
| | | | of 5% (w/v) | of 15% (w/v) |
| | | | cyclodextrin | cyclodextrin |
| Cyclodextrin | Type | CE | (mg/ml) | (mg/ml) |
| α-Cyclodextrin | B_{S} | 0.54 | 0.76 ± 0.016 | 4.22 ± 0.593 |
| 2-Hydroxypropyl- | A_{P} | 0.031 | 0.084 | 0.46 |
| α-cyclodextrin | | | | |
| γ-Cyclodextrin | A_{L} | 0.0049 | 0.062 ± 0.0006 | 0.11 ± 0.0015 |
| 2-Hydroxypropyl- | A_{L} | 0.0011 | 0.048 ± 0.004 | 0.062 ± 0.005 |
| γ-cyclodextrin | | | | |
| β-Cyclodextrin | A_{L} | 0.030 | Not determined | Not determined |
| Randomly | A_{P} | 0.053 | 0.13 | 0.76 |
| methylated | | | | |
| β-cyclodextrin | | | | |

Results of the solubility studies. Mean of three determinations±standard deviation. The solubility of cyclosporin A in pure water under these same conditions was determined to be 0.043±0.004 mg/ml.

The results in Table 1 show that cyclodextrins have a solubilizing effect on cyclosporin A, and the solubility increases with increasing cyclodextrin concentration in the aqueous media. α-Cyclodextrin has a greater solubilizing effect on cyclosporin A and displays higher CE than the other cyclodextrins tested. The solubility of cyclosporin A was shown to be 4.2 mg/ml in pure aqueous solution containing 15% (w/v) α-cyclodextrin. The phase-solubility diagram was of the type B_{S} (Higuchi, T., Connors, K. A., 1965. Phase solubility techniques. Advanced Analytical Chemistry of Instrumentation 4, 117-212). The solubility of the natural β-cyclodextrin in pure water at room temperature is only 2% (w/v), and 0.094 mg/ml is the maximum solubility of cyclosporin A in an aqueous 2% (w/v) β-cyclodextrin solution. For γ-cyclodextrin and 2-hydroxypropyl-γ-cyclodextrin, the highest solubility was estimated to be 0.14 mg/ml at 20% (w/v) for γ-cyclodextrin and 0.066 mg/ml at 20% for 2-hydroxypropyl-γ-cyclodextrin. The highest concentration of 2-hydroxypropyl-α-cyclodextrin and randomly methylated-β-cyclodextrin tested was 15% (w/v) and, at that cyclodextrin concentration, the cyclosporin A solubility was determined to be 0.46 mg/ml and 0.72 mg/ml, respectively. α-Cyclodextrin was selected for further development since it displayed much greater solubilizing effect towards cyclosporin A than the other cyclodextrins tested. γ-Cyclodextrin was also tested further due to its superior ability to form nanoparticles.

The quantitative analysis of cyclosporin A was performed on a reversed-phase high-performance liquid chromatography component system Ultimate 3000 Series from Dionex Softron GmbH (Germering, Germany) consisting of a DGP-3600A pump, SRD-3600 solvent rack and degasser, WPS-3000TLS well plate sampler, TCC-3100 column compartment, photodiode array detector and Phenomenex Luna C-18 150 mm×4.60 mm and 5 micron column, with a matching guard column. The mobile phase consisted of acetonitrile, methanol and water (60:20:20), the flow rate was 1 ml/min, the column oven temperature was 80° C. and the detection wavelength was 205 nm.

### Example 2

The cyclosporin A fraction present in cyclosporin A/cyclodextrin aggregates in the aqueous eye drop media was determined. The aqueous 0.05% (w/v) cyclosporin A eye drop microsuspensions were prepared by dissolving benzalkonium chloride (20 mg) and disodium edetate dehydrate (100 mg) in 70 ml aqueous 1.4% (w/v) polyvinyl alcohol solution. Then 50 mg of cyclosporin A and measured amounts of the different cyclodextrins (i.e., pure α-cyclodextrin, pure γ-cyclodextrin or mixtures of α-cyclodextrin and γ-cyclodextrin) were added to the solution and it was shaken until a homogenous suspension was obtained. The volume was then adjusted to 100.0 ml with aqueous 1.4% (w/v) polyvinyl alcohol solution and heated in a sealed container in an autoclave at 121° C. for 20 min. The suspension was cooled down to room temperature under sonication. Then, the suspension was removed from the sonicator and allowed to equilibrate at room temperature under constant agitation for 7 days, protected from light. Compositions of the different aqueous cyclosporin A eye drop suspensions tested (F1 to F7) are listed in Table 2. Eye drop formulation no. 5 (F5) was also prepared without some of the excipients in an effort to evaluate the excipient effects on the cyclodextrin solubilization of cyclosporin A and aggregation of the cyclosporin A/cyclodextrin complexes. The composition of these eye drops (F8 to F10) is given in Table 3.

TABLE 2

| | γ-Cyclodextrin | α-Cyclodextrin |
|---|---|---|
| Formulation | (% w/v) | (% w/v) |
| F1 | 15.0 | 0.00 |
| F2 | 13.0 | 1.00 |
| F3 | 12.0 | 2.00 |
| F4 | 11.0 | 3.00 |
| F5 | 10.0 | 4.00 |
| F6 | 9.00 | 5.00 |
| F7 | 0.00 | 5.00 |

Composition of cyclosporin A eye drop formulations. In addition to cyclodextrins, each formulation contained 0.05% (w/v) cyclosporin A, 1.4% (w/v) polyvinyl alcohol, 0.02% (w/v) benzalkonium chloride and 0.1% (w/v) disodium edetate dehydrate.

TABLE 3

| Formulation | Excipients |
|---|---|
| F8 | Without excipients |
| F9 | 1.4% (w/v) polyvinyl alcohol |
| F10 | 0.020% (w/v) benzalkonium chloride and 0.10% |
| | (w/v) EDTA |

Composition of the test formulations. Each formulation contained, in addition to the listed excipients, 0.05% (w/v) cyclosporin A, 10% (w/v) γ-cyclodextrin and 4% (w/v) α-cyclodextrin.

The formulation (4 ml) being tested was centrifuged at 6000 rpm at room temperature (22-23° C.) for 20-30 min. If the formulation separated into two layers, the upper layer was analyzed by high-performance liquid chromatography (see EXAMPLE 1). The drug content in solid phase was calculated as:

$$\% solid drug fraction = \frac{( Total drug - dissolved drug )}{Total drug content} \times 100$$

The solid drug fraction was determined in each of the seven formulations (Table 4). In addition, formulation F5 (which contains 10% w/v γ-cyclodextrin and 4% w/v α-cyclodextrin) was also tested with and without the other excipients (i.e., polyvinyl alcohol, benzalkonium chloride and disodium edetate dehydrate) to evaluate excipient effect on the aggregation.

TABLE 4

| | Solid drug |
|---|---|
| Formulation | fraction % |
| F1 (15% γ-cyclodextrin) | 80.0 |
| F2 (13% γ-cyclodextrin + 1% α-cyclodextrin) | 62.9 |
| F3 (12% γ-cyclodextrin + 2% α-cyclodextrin) | 44.0 |
| F4 (11% γ-cyclodextrin + 3% α-cyclodextrin) | 34.0 |
| F5 (10% γ-cyclodextrin + 4% α-cyclodextrin) | 28.8 |
| F6 (9% γ-cyclodextrin + 5% α-cyclodextrin) | 30.6 |
| F7 (5% α-cyclodextrin) | 11.0 |
| F8 (F5 without polyvinyl alcohol, benzalkonium chloride and | 34.3 |
| disodium edetate dehydrate) | |
| F9 (F5 with only 1.4% polyvinyl alcohol) | 36.8 |
| F10 (F5 with only benzalkonium and disodium edetate | 44.0 |
| dehydrate) | |

Solid drug fraction in the different cyclosporin A formulations.

For formulation F7, which contains only α-cyclodextrin, the solid drug fraction was low and most of the drug was in the liquid phase. When the formulation contains a mixture of γ-cyclodextrin and α-cyclodextrin, the solid drug fraction increases, and when the formulation contains only γ-cyclodextrin, most of the drug is in the solid phase. Formulations F1, F2 and F3, which contain the lowest amount of α-cyclodextrin and the highest of γ-cyclodextrin, separated into 3 layers when centrifuged, where the top layer contains only cyclosporin A. This indicates that some of the drug did not form complexes with cyclodextrin and did therefore not dissolve in the aqueous media. Other formulations separated into 2 layers during centrifugation, which shows that a formulation must contain at least 3% α-cyclodextrin to fully dissolve cyclosporin A. In formulations F4, F5 and F6 more drug was in the solid phase than in formulation F7, but all cyclosporin A appeared to have been dissolved. This means that some cyclosporin A is in cyclosporin A/cyclodextrin complexes and that the complexes formed have aggregated into particles that precipitated during centrifugation. This also shows that γ-cyclodextrin increases the aggregation.

The excipient effect on the aggregation was also investigated. Formulation F5, which contains 10% (w/v) γ-cyclodextrin and 4% (w/v) α-cyclodextrin, was selected for these studies in which all cyclosporin A is dissolved and in which the solid drug fraction was within a suitable range. For formulation F8, which contained cyclodextrin but no excipients, the solid drug fraction was similar to a formulation containing all the excipients, like F5. In formulation F9, which contained cyclodextrin and polyvinyl alcohol, the aggregation was slightly increased. The largest solid drug fraction was found in formulation F10, which contained cyclodextrin as well as both benzalkonium chloride and disodium edetate dehydrate but no polyvinyl alcohol. This shows that the excipients have some effect on the aggregation.

The physiochemical properties of F1, F5 and F7 were determined. The pH values of the formulations were determined at room temperature (22-23° C.). Viscosity measurements of the eye drops formulations were performed with a Brookfield model DV-I⁺ (USA) viscometer at 25±2° C., and the osmolality of the formulations was determined in a vapor pressure osmometer operated at 25° C. (TABLE 5).

TABLE 5

| Formulation | F1 | F5 | F7 |
|---|---|---|---|
| pH value | 5.03 | 5.24 | 5.33 |
| Viscosity | 3.49 ± 0.0770 cP | 3.86 ± 0.0160 cP | 2.23 ± 0.0159 cP |
| Osmolality | 129 ± 1.04 | 127 ± 2.34 | 67.4 ± 0.881 |
| | mOsm/kg | mOsm/kg | mOsm/kg |

Viscosity, pH values and osmolality of formulations F1, F5 and F7. Mean of three determinations ±SD.

### Example 3

The particle size characterization of the eye drop formulations was performed by dynamic light scattering (DLS). Each formulation was filtered through a 0.45 μm membrane filter before the measurements (to exclude particles larger than 0.45 μm) that were carried out at 25° C., 180° scattering angle and a 780 nm laser beam, and each measurement was done in triplicate. Particle sizes were also determined visually using a light microscope without sample filtration, which gives a better idea of how many particles there are in the suspension and how large they are. TABLE 6, shows the size distribution data from DLS measurements.

TABLE 6

| Formulation | d (nm) | Width (nm) | % I |
|---|---|---|---|
| F1 | 203.0 | 95 | 28.7 |
| | 370.0 | 160 | 70 |
| | 1302 | 288 | 1.30 |
| F2 | 412.0 | 199 | 96 |
| | 1534 | 497 | 4.0 |
| F3 | 375.0 | 230 | 95.4 |
| | 1191 | 407 | 4.6 |
| F4 | 4.96 | 4.0 | 23 |
| | 223 | 140 | 66.3 |
| | 588 | 279 | 8.42 |
| | 2103 | 573 | 2.26 |
| F5 | 1.10 | 0.43 | 4.10 |
| | 5.14 | 4.25 | 29.5 |
| | 244.3 | 139 | 64.2 |
| | 731 | 37.8 | 2.26 |
| F6 | 1.18 | 0.6 | 2.58 |
| | 5.34 | 4.27 | 25.3 |
| | 147 | 53.1 | 9.90 |
| | 252 | 110 | 50.9 |
| | 436.3 | 147 | 8.90 |
| | 918 | 305 | 3.48 |
| F7 | 6.32 | 4.9 | 27.4 |
| | 155 | 79.3 | 72.7 |
| F8 | 33.1 | 12.2 | 0.34 |
| | 156.1 | 138 | 99.7 |
| F9 | 1.22 | 1.3 | 5.34 |
| | 5.37 | 5.2 | 26.4 |
| | 201 | 132 | 48.4 |
| | 434 | 185 | 15.5 |
| | 850 | 289 | 4.4 |
| F10 | 1.53 | 0.73 | 12.8 |
| | 246 | 161 | 75.3 |
| | 362 | 125 | 5.28 |
| | 611 | 163 | 6.62 |

The DLS results of cyclosporin A/cyclodextrin complexes size analysis for formulation F1-F10, data reported as hydrodynamic diameter (d) in nano-scale range, width of the population and intensity distribution (% I)

DLS measurement of formulations F1, F2 and F3 gave 2 or 3 size populations and the mean diameter was 200-400 nm based on the intensity distribution. When observed by microscope, these three formulations contained greater amount of relatively larger particles than the other formulations. This was mainly due to the presence of solid drug particles and not due to aggregation of drug/cyclodextrin complexes.

In formulations F4, F5 and F6, cyclosporin A is essentially completely dissolved and the size distributions were greater than in the other formulations, several size populations were detected and the main particle sizes were determined to be 4.9-5.3 nm and 150-250 nm. In formulation F7, which contains only α-cyclodextrin, two size populations occur where the main particle sizes were 6 nm and 155 nm. When this formulation was measured by a light microscope, the formulation appeared clear. These results indicate that when the formulation contains only α-cyclodextrin, the cyclosporin A/cyclodextrin complexes do not have a strong tendency to form larger aggregates. When the formulation contains both α-cyclodextrin and γ-cyclodextrin the complexes have stronger tendencies to form aggregates and the aggregates formed are also larger.

The excipient effect on the aggregation was tested in formulations F8, F9 and F10. When measured by a light microscope, all of these formulations appeared mostly clear with a very few large 1-5 μm particles. Only when observed by DLS, some differences could be detected. In formulation F8, where no excipients were included except the cyclodextrins, two size populations were detected with main particle sizes at 33.1 nm and 156 nm. When the formulation contained benzalkonium chloride and disodium edetate dehydrate (F10), four size populations occur with main particle size determined to be 1.53, 246, 362 and 611 nm. When the formulation contained only polyvinyl alcohol (F9), five size populations were detected with main particle size of 1.22, 5.37, 201, 434 and 850 nm. These results indicate that the excipients increase the aggregate formation in the aqueous eye drop media.

### Example 4

Transmission electron microscope (TEM) is the analytical method of choice to detect the morphology and sizes of drug/cyclodextrin complexes including of their aggregates. The morphology and size of aggregates in selected cyclosporin A aqueous eye drop suspensions (i.e., F1, F5 and F7) were analyzed. Initially, the samples were centrifuged at 4000 rpm, 20° C. for 30 min (Model Rotina 35R, Hettuch, Germany), then the supernatant was pipetted off and formvar-coated grids were floated on a droplet of the preparation on Parafilm®, to permit the absorption of the nanoparticles onto the grid. After blotting the grid with a filter paper, the grid was transferred onto a drop of the negative stain by using aqueous uranyl acetate solution (1%) under constant vacuum. Finally, the samples were examined in a Model JEM-2100 transmission electron microscope (JEOL, Tokyo, Japan). TEM micrographs of the selected cyclosporin A eye drops suspensions show spherical aggregates of cyclosporin A/cyclodextrin complexes with the diameter of 40-140 nm and 20-100 nm in F1 and F5, respectively. The dominant sizes of cyclosporin A/cyclodextrin aggregated nanoparticles in F7 were less than 10 nm. However, small amounts of larger particles (120-140 nm) were also detected. The aggregate size of TEM monographs was in accordance with the DLS technique. Observation of spherical aggregates indicates that the aggregates of cyclosporin A/cyclodextrin complexes can enhance drug solubility through non-inclusion complexes and/or micelle-like structures. The diameter of the assembled nanoparticles in F5 ranged from 100 to 400 nm. The particle sizes observed by TEM are in agreement with those obtained by DLS.

The morphology of F5 was further analyzed using a scanning electron microscope (SEM). After gentle agitation the solid material of the aqueous eye drop suspension was layered on a slide, and the sample allowed to dry overnight in a desiccator at room temperature. Subsequently, this layer was coated with gold under an argon atmosphere at room temperature. Samples were then observed for their surface morphology with a SEM (Model JSM-5410LV, JEOL, Tokyo, Japan). SEM showed nanoparticles with a diameter from 100 to 400 nm which is in agreement with the DLS and TEM results.

### Example 5

The permeation of cyclosporin A from F1, F5 and F7 through a series of semi-permeable membranes was measured in a Franz diffusion cell apparatus consisting of a donor and a receptor compartment. The donor and receptor chambers were separated by a single layer of semi-permeable membrane with MWCO of 20, 50 or 100 kDa and diffusion area of 1.77 cm². The membranes were soaked in Milli-Q water over night prior to the permeation studies. The donor phases consist of 2 ml of the formulation to be tested (i.e., F1, F5 or F7). Receptor phase (12 ml) for formulation F7 consisted of formulation F7 without cyclosporin A and polyvinyl alcohol, and the receptor phases for formulations F1 and F5 consisted of formulation F5 without cyclosporin A and polyvinyl alcohol. This is due to the fact that at least 3% (w/v) of α-cyclodextrin is needed to dissolve 0.5 mg/ml of cyclosporin A, and that formulations F1 and F5 have similar osmolality. Polyvinyl alcohol was excluded from the receptor phases due to the fact that the polyvinyl alcohol sticks to the flow cell, resulting in a low UV light density and poor HPLC measurements. The receptor phase was degassed to remove dissolved air before it was placed in the receptor compartment. The study was carried out at room temperature under continuous stirring of the receptor phase by a magnetic stirring bar rotating at 300 rpm. A 100 μl sample of the receptor media was withdrawn at 5, 6, 7, 8 and 9 hours and replaced immediately with fresh receptor phase. The cyclosporin A concentration in the receptor sample was measured by HPLC (see EXAMPLE 1). The flux (J) was calculated from the slope (dq/dt) of the linear section of the permeation profiles, that is, the amount of cyclosporin A in the receptor chamber (q) versus time (t) profiles, and the permeability coefficient (P_{C}) was calculated from the flux:

$$J = \frac{dq}{A \cdot dt} = {P}_{C} \cdot {C}_{d}$$

where A is the surface area of the membrane (1.77 cm²) and C_{d} is the initial concentration of dissolved cyclosporin A in the donor phase.

The molecular weight of cyclosporin A is 1202.6 Da and the molecular weights of α-cyclodextrin and γ-cyclodextrin are 972.84 and 1297.12 Da, respectively. Monomeric cyclosporin A molecules and cyclosporin A/cyclodextrin (1:1) complexes are able to penetrate easily through these membranes. The study shows that cyclosporin A is mainly present as cyclosporin A/cyclodextrin complexes that have aggregated into particles with diameter greater than 20 kDa and could therefore not penetrate the MWCO 20 kDa membrane. For formulation F1, which contains only γ-cyclodextrin, the limited amount of dissolved drug in the donor media could also be the reason for this lack of detection in the receptor phase, since the drug must be dissolved to penetrate the membrane. Cyclosporin A in all three formulations penetrated membranes with MWCO 50 and 100 kDa, showing that most of the aggregates are smaller than 50 kDa. The flux and permeability coefficient for each formulation were calculated (TABLE 7). Formulation F5 and F7 gave similar flux values, but formulation F1 gave lower flux values. Again, this is mainly due to the lower concentration of dissolved cyclosporin A in the donor media and the fact that only dissolved cyclosporin A, free or in cyclodextrin complexes, can penetrate through the membranes.

TABLE 7

| | MWCO 50.000 Da | | MWCO 100.000 Da | |
|---|---|---|---|---|
| Formulation | Flux (μg/h/cm²) | Pc (cm/h) | Flux (μg/h/cm²) | Pc (cm/h) |
| F1 | 6.58 | 6.58 × 10⁻² | 7.28 | 7.28 × 10⁻² |
| F5 | 27.8 | 5.57 × 10⁻² | 28.8 | 5.76 × 10⁻² |
| F7 | 32.1 | 6.41 × 10⁻² | 28.0 | 5.60 × 10⁻² |

Flux and permeability coefficient (Pc) for cyclosporin A in formulations F1, F5 and F7 through semi permeable membranes with MWCO 50.000 and 100.000 Da.

### Example 6

The cyclosporin A/drug aggregates behavior was studied further. Small samples of F5 were filtered through a 0.45 μm membrane filter-diluted with an equal volume of the mobile phase or only centrifuged at 6000 rpm at room temperate for 20-30 min. The cyclosporin A concentrations of the solutions obtained were then determined by HPLC (see EXAMPLE 1). The filtered formulation was also allowed to stand for one day and then centrifuged at room temperature for 20-30 min and the cyclosporin A concentration determined by HPLC. When the sample is diluted, all of the aggregates are dissolved and the cyclosporin A concentration represents the total amount of drug in the suspension. When the suspension is filtered, the concentration of cyclosporin A in the filtrate should be close to or the same as when the suspension is centrifuged, since most of the aggregates should be filtered from the solution just like during centrifugation. This was not the case, however, and the cyclosporin A concentration of the filtered suspension was close to the diluted one, not to the centrifuged one (TABLE 8). Also, when the suspension is filtered, it becomes transparent, but, interestingly, when the filtered suspension had been standing for one day, some aggregation occurred and the solution became again non-transparent. Therefore, the filtered formulation was centrifuged after standing at room temperature for one day and then the concentration of dissolved cyclosporin A was measured. Then the cyclosporin A concentration was close to the centrifuged one, not to the diluted one. This indicates that the cyclosporin A/cyclodextrin complexes are aggregating, but the forces holding these complexes together in aggregates are very weak and break during filtration but are then reformed in the filtrate.

TABLE 8

| | Diluted with | Filtered | Filtered | |
|---|---|---|---|---|
| | Mobile | through | and then | |
| | phase | 0.45 | centrifuge | |
| | (1:1) | μm filter | after 1 day. | Centrifuge |
| Concentration of | 0.462 | 0.430 | 0.383 | 0.360 |
| cyclosporin A | | | | |
| (mg/ml) | | | | |

Concentration of cyclosporin A in formulation F5 after being diluted, centrifuged, filtered, filtered and centrifuged after 1 day.

### Example 7

Three eye drop formulations were prepared and their physiochemical properties determined as described in EXAMPLE 2 (TABLE 9). The amounts of α-cyclodextrin present in the eye drops, that is, 4%, 12.5% and 15%, were more than sufficient to solubilize all cyclosporin A present in the eye drop formulations, that is, 0.05%, 0.2% and 0.4%, respectively. The aqueous solubility of α-cyclodextrin and γ-cyclodextrin is 13% and 25% (w/v), respectively. Small nanoparticles were formed, and the aqueous eye drops became opalescent, upon addition of γ-cyclodextrin. The eye drops were centrifuged at relatively low speed that only removed the larger particles (in the low microparticle range) from the eye drop suspension while the nanoparticles remained in solution (EXAMPLE 3). The solid fraction, which consisted of cyclosporin A/cyclodextrin complex microparticles, was between 23% and 29%.

TABLE 9

| | A | B | C |
|---|---|---|---|
| Cyclosporin A | 0.050% | 0.20% | 0.40% |
| α-Cyclodextrin | 4.0% | 12.5% | 15.0% |
| γ-Cyclodextrin | 10.0% | 12.5% | 15.0% |
| Polyvinyl alcohol (PVA) | 1.4% | 1.6% | 1.6% |
| Disodium edetate dehydrate (EDTA) | 0.10% | 0.10% | 0.10% |
| Benzalkonium chloride (BAC) | 0.02% | 0.02% | 0.02% |
| Purified water | q.s. | q.s. | q.s. |
| Viscosity | 3.9 cP | 5.5 cP | 7.0 cP |
| pH | 5.2 | 5.4 | 5.4 |
| Osmolarity | 129 | 284 | 366 |
| | mOsm/kg | mOsm/kg | mOsm/kg |
| Solid drug fraction in cyclosporin | 29% | 25% | 23% |
| A/CD complex | | | |

Compositions of Eye Drops

Two healthy volunteers (a male and a female, both 30 years old) received one drop of formulation B in the left eye. No burning sensation, blurred vision, itching or other side effects were observed. The eye drops were well tolerated. Formulation B contained 4 times the 0.05% concentration of cyclosporin A which is sold commercially as Restasis® eye drops. Further testing is expected to determine that even higher concentrations, such as 0.50%, are also well tolerated.

Although not wishing to be bound by any particular theory or mechanism, it is believed that the aqueous eye drops described herein provide a larger and more effective amount of cyclosporin, particularly cyclosporin A, per dose, which can, in a regular dosing regimen, for example twice per day, more quickly and more thoroughly achieve production of tears sooner and more effectively than otherwise possible with the lower dose oil-based marketed product. This can be due to better penetration into the lacrimal apparatus, including the lacrimal glands, which secrete tears and their excretory ducts, which convey the tear fluid to the surface of the eye to cover the conjunctiva and cornea. It can also be due to the sustained release which the subject eye drops provide of the cyclosporin and the protective and soothing effect which the agglomerates provide on the eye surface.

Although this description has been couched in some detail by way of illustration, EXAMPLES and preferred embodiments, for purposes of clarity of understanding, it will be appreciated by one of ordinary skill that various modifications, substitutions, omissions and changes can be made without departing from the spirit thereof. Accordingly, it is intended that the scope hereof is limited solely to the scope of the following claims, and equivalents thereof.

What is claimed is:

1. An aqueous ophthalmic composition comprising:
(a) cyclosporin A in an amount which is effective ophthalmologically;
(b) α-cyclodextrin in an amount effective to form a water-soluble complex with said cyclosporin A;
(c) γ-cyclodextrin in an amount effective to produce formation of cyclosporin A/α-cyclodextrin complex aggregates;
(d) cyclosporin A/cyclodextrin particles with diameters of from about 100 nm to about 100 μm, comprising both said α-cyclodextrin and said γ-cyclodextrin;
(e) water; and
(f) optionally, a polymeric stabilizing agent;
the total concentration of said cyclosporin A in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from 1% (w/v) to about 25% (w/v), and the total fraction of cyclosporin in particles with diameters greater than about 300 nm being not less than about 10%.

2. The ophthalmic composition of claim 1, wherein the stabilizing agent is selected from the group consisting of polyoxyethylene fatty acid esters, polyoxyethylene alkylphenyl ethers, and polyoxyethylene alkyl ethers.

3. The ophthalmic composition of claim 1, wherein the stabilizing agent is a polymer selected from the group consisting of water-soluble cellulose derivatives, carboxyvinyl polymers, polyvinyl polymers, polyvinyl alcohols and polyvinylpyrrolidones.

4. The ophthalmic composition of claim 1, wherein:
(a) cyclosporin A is present in an amount of from about 0.05% (w/v) to about 1.0% (w/v);
(b) α-cyclodextrin is present in an amount of from about 4% (w/v) to about 20% (w/v);
(c) γ-cyclodextrin is present in an amount of from about 4% (w/v) to about 25% (w/v); and
(d) the solid drug fraction comprises cyclosporin/cyclodextrin particles with diameters from about 200 nm to about 50 μm.

5. A method of inducing or enhancing tear formation in a subject in need thereof, said method comprising topically administering to the eye or eyes of said subject an amount of a composition of claim 1 effective to induce tear formation.

6. The method of claim 5, wherein the subject is suffering from dry eye.

7. A method of forming agglomerates of cyclosporin A, said method comprising solubilizing a therapeutically effective amount of cyclosporin A, in water, in a quantity of α-cyclodextrin sufficient to essentially completely dissolve said cyclosporin A, and in sufficient γ-cyclodextrin to form cyclosporin A/α-cyclodextrin complex aggregates, optionally with a polymeric stabilizing agent, to produce cyclosporin A/cyclodextrin particles with diameters of from about 100 nm to about 100 μm, comprising both said α-cyclodextrin and said γ-cyclodextrin.

8. An aqueous ophthalmic composition comprising:
(a) cyclosporin A in an amount which is therapeutically effective ophthalmologically;
(b) α-cyclodextrin in an amount effective to form a water-soluble complex with said cyclosporin A;
(c) γ-cyclodextrin in an amount effective to produce formation of cyclosporin A/α-cyclodextrin complex aggregates;
(d) cyclosporin A/cyclodextrin particles with diameters of from about 100 nm to about 100 μm, comprising both said α-cyclodextrin and said γ-cyclodextrin;
(e) water; and
(f) optionally, a polymeric stabilizing agent;
the total concentration of said cyclosporin A in the composition being from about 0.01% (w/v) to about 1.0% (w/v), the total concentration of said α-cyclodextrin in the composition being from about 1% (w/v) to about 25% (w/v), the total γ-cyclodextrin concentration in the composition being from about 1% (w/v) to about 25% (w/v), the total fraction of cyclosporin A in particles with diameters greater than about 300 nm being not less than about 10%, for use in eliciting the formation of tears by topical administration of an effective amount thereof to the eye or eyes of a subject in need of tear formation.

9. The composition of claim 8, wherein the subject is suffering from dry eye.

10. Use of cyclosporin A in the manufacture of an aqueous ophthalmic composition comprising:
(a) cyclosporin A in an amount which is therapeutically effective ophthalmologically;
(b) α-cyclodextrin in an amount effective to form a water-soluble complex with said cyclosporin A;
(c) γ-cyclodextrin in an amount effective to produce formation of cyclosporin A/α-cyclodextrin complex aggregates;
(d) cyclosporin A/cyclodextrin particles with diameters of from about 100 nm to about 100 μm, comprising both said α-cyclodextrin and said γ-cyclodextrin;
(e) water; and
(f) optionally, a polymeric stabilizing agent;
the total concentration of cyclosporin A in the composition being from about 0.01% (w/v) to about 1.0% (w/v), the total concentration of α-cyclodextrin in the composition being from about 1% (w/v) to about 25% (w/v), the total γ-cyclodextrin concentration in the composition being from about 1% (w/v) to about 25% (w/v), the total fraction of cyclosporin A in particles with diameters greater than about 300 nm being not less than about 10%, for eliciting tear formation by topical administration of an effective amount thereof to the eye or eyes of a subject in need of tear formation.

11. Use of claim 10, wherein the subject is suffering from dry eye.

12. The aqueous ophthalmic composition of claim 8, wherein the stabilizing agent is selected from the group consisting of polyoxyethylene fatty acid esters, polyoxyethylene alkylphenyl ethers, and polyoxyethylene alkyl ethers.

13. The aqueous ophthalmic composition of claim 8, wherein the stabilizing agent is a polymer selected from the group consisting of water-soluble cellulose derivatives, carboxyvinyl polymers, polyvinyl polymers, polyvinyl alcohols and polyvinylpyrrolidones.

14. The aqueous ophthalmic composition of claim 8, wherein:
(a) cyclosporin A is present in an amount of from about 0.05% (w/v) to about 1.0% (w/v);
(b) α-cyclodextrin is present in an amount of from about 4% (w/v) to about 20% (w/v);
(c) γ-cyclodextrin is present in an amount of from about 4% (w/v) to about 25% (w/v); and
(d) the solid drug fraction comprises cyclosporin/cyclodextrin particles with diameters from about 200 nm to about 50 μm.

15. Use according to claim 10, wherein the stabilizing agent is selected from the group consisting of polyoxyethylene fatty acid esters, polyoxyethylene alkylphenyl ethers, and polyoxyethylene alkyl ethers.

16. Use according to claim 10, wherein the stabilizing agent is a polymer selected from the group consisting of water-soluble cellulose derivatives, carboxyvinyl polymers, polyvinyl polymers, polyvinyl alcohols and polyvinylpyrrolidones.

17. Use according to claim 10, wherein:
(a) cyclosporin A is present in an amount of from about 0.05% (w/v) to about 1.0% (w/v);
(b) α-cyclodextrin is present in an amount of from about 4% (w/v) to about 20% (w/v);
(c) γ-cyclodextrin is present in an amount of from about 4% (w/v) to about 25% (w/v); and
(d) the solid drug fraction comprises cyclosporin/cyclodextrin particles with diameters from about 200 nm to about 50 μm.
